(19) United States
(12) Patent Application Publication
(10) Pub. No.: US 2019/0218521 A1
(43) Pub. Date: Jul. 18, 2019

(54) Culture medium of viruses for human vaccines have to consist of human cells from placenta and/or from umbilical cord of a fetus of the blood type 0 Rh- and of the mother of the blood type 0 Rh- (both, fetus and mother, must be of the blood type 0 Rh-)

(71) Applicant: Lorenzo Bracco, Torino (IT)

(72) Inventor: Lorenzo Bracco, Torino (IT)

(21) Appl. No.: 15/874,030

(22) Filed: Jan. 18, 2018

(51) Int. Cl. C12N 7/00

(52) U.S. Cl. CPC C12N 7/00; C12N 2500/84; C12N 2700/00

(57) ABSTRACT

The culture medium of viruses for human vaccines have to consist of human cells from placenta and/or from umbilical cord of a fetus of the blood type 0 Rh− (blood type 0 Rh negative) and of the mother of the blood type 0 Rh− (both, fetus and mother, must be of the blood type 0 Rh−).

A replicating virus needs to infect replicating cells. It is not obvious to know in detail what cell culture is done to replicate viruses from which different antigens for different vaccines are obtained, being the procedure one of those practices largely covered by industrial secret. As far as I have been able to get information, they are human cells, from human fetus, replicating for decades.

When those cells were harvested, was the fetal and mother blood type considered? I understand that human fetal cells, duly washed, should not contain blood type antigens, but are we sure that these antigens have not remained in tiny traces that can reactivate a highly activated immune system? I refer to those cases of abnormal activation comparable to the strongest allergies. For example in cheese allergy, in extreme cases, just entering and breathing in a room where cheese had been preserved in the past can trigger a very serious crisis. If the blood type of the fetus from which the cells were taken for the cultivation of pathogenic agents for vaccines were not checked, the risk would increase considerably if, instead of using a monovalent vaccine for a single pathogen, such as measles, we use combined vaccines that simultaneously vaccinate for multiple pathogens. Each vaccine coming from a different culture of fetal cells may increase the risk of blood type antigen A or B. This risk is obviously increasing as much as the vaccine is polyvalent because each viral agent will be cultivated on its own culture medium.

Conclusion, what do I propose for the production of vaccines as a “modus operandi” safer than now?

Vaccines for human use for viral diseases: each species of virus from which the vaccine is obtained is grown on a culture medium of living cells in replication. The culture medium of viruses for human vaccines have to consist of human cells from placenta and/or from umbilical cord of a fetus of the blood type 0 Rh− (blood type 0 Rh negative) and of the mother of the blood type 0 Rh− (both, fetus and other, must be of the blood type 0 Rh−).

1. The culture medium of viruses for human vaccines have to consist of human cells from placenta and/or from umbilical cord of a fetus of the blood type 0 Rh− (blood type 0 Rh negative) and of the mother of the blood type 0 Rh− (both, fetus and mother, must be of the blood type 0 Rh−).
